(19) United States
(12) Patent Application Publication
(10) Pub. No.: US 2024/0416126 A1
(43) Pub. Date: Dec. 19, 2024

(54) MACHINE LEARNING FOR TREATMENT OF PHYSIOLOGICAL DISORDERS

(71) Applicant: Cochlear Limited, Macquarie University, NSW (AU)

(72) Inventor: Kelly ASSOULY, Brussels (BE)

(21) Appl. No.: 18/687,147

(22) PCT Filed: Aug. 12, 2022
(86) PCT No.: PCT/IB2022/057559

§ 371 Date: Feb. 27, 2024

Related U.S. Application Data
(60) Provisional application No. 63/240,421, filed on Sep. 3, 2021.

(51) Int. Cl. A61N 1/36

(52) U.S. Cl. CPC A61N 1/361; A61N 1/36038; A61N 1/36139

(57) ABSTRACT

Presented herein are techniques for use of machine learning for treatment of physiological disorders, including for detection of a physiological event and adaption operation of an implantable medical device system to acutely treat the physiological event.

### BACKGROUND

### Field of the Invention

The present invention relates generally to the use of machine learning for treatment of physiological disorders.

### Related Art

Medical devices have provided a wide range of therapeutic benefits to users over recent decades. Medical devices can include internal or implantable components/devices, external or wearable components/devices, or combinations thereof (e.g., a device having an external component communicating with an implantable component). Medical devices, such as traditional hearing aids, partially or fully-implantable hearing prostheses (e.g., bone conduction devices, mechanical stimulators, cochlear implants, etc.), pacemakers, defibrillators, functional electrical stimulation devices, and other medical devices, have been successful in performing lifesaving and/or lifestyle enhancement functions and/or monitoring for a number of years.

The types of medical devices and the ranges of functions performed thereby have increased over the years. For example, many medical devices, sometimes referred to as “implantable medical devices,” now often include one or more instruments, apparatus, sensors, processors, controllers or other functional mechanical or electrical components that are permanently or temporarily implanted in a user. These functional devices are typically used to diagnose, prevent, monitor, treat, or manage a disease/injury or symptom thereof, or to investigate, replace or modify the anatomy or a physiological process. Many of these functional devices utilize power and/or data received from external devices that are part of, or operate in conjunction with, implantable components.

### SUMMARY

In one aspect, a tinnitus therapy apparatus is provided. The tinnitus therapy apparatus comprises: a stimulation component configured to deliver stimulation signals to at least one user; and a machine learning device that detects onset of tinnitus events of the least one user with respect to an external sound environment, wherein the machine learning device includes: a state observing unit that obtains state data comprising physiological data representing a current physiological state of the at least one user and environmental data representing an external sound environment of the at least one user, a label data unit that acquires label data associated with the onset of tinnitus events, and a learning unit that, by using the state data and the label data, detects the onset of the tinnitus events of the at least one user and generates device configuration data, wherein the device configuration data indicates a tinnitus therapy for delivery to the at least one user via the stimulation component.

In another aspect, a method for treating tinnitus events using machine learning is provided. The method comprises: obtaining, with a state observing unit, state data indicating a current physiological state of at least one user; obtaining, with a label data unit, label data associated with onset of tinnitus events; and using the state data and the label data in a machine-learning model to automatically detect onset of tinnitus events of the at least one user and generate device configuration data indicates a tinnitus therapy for delivery to the at least one user, wherein the state observing unit, the label data unit, and the machine-learning model comprise one or more of logic hardware and a non-transitory computer readable medium storing computer executable code within a tinnitus therapy system.

In another aspect, an apparatus is provided. The apparatus comprises: a stimulation component configured to deliver stimulation signals to at least one user; and a machine learning device that detects onset of physiological events of the least one user with respect to an external environment of the at least one user, wherein the machine learning device includes: a state observing unit that obtains state data comprising physiological data representing a current physiological state of the at least one user and environmental data representing the external environment of the at least one user, a label data unit that acquires label data associated with the onset of physiological events, a learning unit that, by using the state data and the label data, detects the onset of the physiological events of the at least one user and generates device configuration data, wherein the device configuration data indicates a therapy for delivery to the at least one user via the stimulation component.

In another aspect, one or more non-transitory computer readable storage media are provided. The non-transitory computer readable storage media comprise instructions that, when executed by a processor, cause the processor to: obtain, with a state observing unit, state data indicating a current physiological state of at least one user; obtain, with a label data unit, label data associated with onset of physiological events; and use the state data and the label data in a machine-learning model to automatically detect onset of physiological events of the at least one user and generate device configuration data indicates a therapy for delivery to the at least one user.

### BRIEF DESCRIPTION OF THE DRAWINGS

Embodiments of the present invention are described herein in conjunction with the accompanying drawings, in which:

FIG. 1A is a schematic diagram illustrating a cochlear implant system with which aspects of the techniques presented herein can be implemented;

FIG. 1B is a side view of a user wearing a sound processing unit of the cochlear implant system of FIG. 1A;

FIG. 1C is a schematic view of components of the cochlear implant system of FIG. 1A;

FIG. 1D is a block diagram of the cochlear implant system of FIG. 1A;

FIG. 2 is a functional block diagram of a machine-learning therapy device, in accordance with certain embodiments presented herein techniques presented herein;

FIGS. 3A, 3B, and 3C are example user interfaces for use with a machine-learning therapy device for treatment of tinnitus, in accordance with certain embodiments presented herein;

FIG. 4 is a functional block diagram illustrating integration of a machine-learning therapy device within a tinnitus therapy system, in accordance with certain embodiments presented herein;

FIG. 5 is a flowchart of an example method, in accordance with embodiments presented herein; and

FIG. 6 is a schematic diagram illustrating a vestibular implant system with which aspects of the techniques presented can be implemented.

### DETAILED DESCRIPTION

A physiological disorder is an illness that interferes with the way that the functions of the body are carried out. Physiological disorders are generally caused when the normal or proper functioning of the body is affected because the body's organs have malfunctioned, are not working, and/or the actual cellular structures have changed over a period of time causing illness.

Presented herein are techniques for use of machine learning for treatment of physiological disorders, including for detection of a physiological event and adaption operation of an implantable medical device system to acutely treat the physiological event. As used herein, a “physiological event” refers to the onset or presence of a symptom of a physiological disorder, such as the onset/presence of tinnitus, pain, etc. For ease of illustration, the techniques presented herein will generally be described with reference to treatment of inner ear physiological disorders (inner ear disorders) and, in particular, with reference to treatment of tinnitus. However, it is to be appreciated that the techniques presented herein can be used to treat other inner ear disorders (e.g., vertigo, dizziness, etc.) and other types of physiological disorders (e.g., pain disorders, etc.).

Moreover, also for ease of description, the techniques presented herein are primarily described with reference to a cochlear implant systems and/or tinnitus therapy systems. However, it is to be appreciated that the techniques presented herein may also be partially or fully implemented by other types of devices, including implantable medical devices, computing devices, consumer electronic devices, etc. For example, the techniques presented herein may be implemented by other auditory prosthesis systems that include one or more other types of auditory prostheses, such as middle ear auditory prostheses, bone conduction devices, direct acoustic stimulators, electro-acoustic prostheses, auditory brain stimulators, combinations or variations thereof, etc. The techniques presented herein may also be implemented by dedicated tinnitus therapy devices and tinnitus therapy device systems and/or tinnitus therapy devices forming part of another type of device (e.g., part of a hearing device). In further embodiments, the techniques presented herein may also be implemented by, or used in conjunction with, vestibular devices (e.g., vestibular implants), visual devices (i.e., bionic eyes), sensors, pacemakers, drug delivery systems, defibrillators, functional electrical stimulation devices, catheters, seizure devices (e.g., devices for monitoring and/or treating epileptic events), sleep apnea devices, electroporation devices, etc. The techniques presented herein may also be partially or fully implemented by consumer devices, such as tablet computers, mobile phones, wearable devices, etc.

Tinnitus is the perception of noise or “ringing” in the ears which currently affects an estimated 10-15% of the general population, increasing with age. Tinnitus is a common artefact of hearing loss, but can also be a symptom of other underlying conditions, such as ear injuries, circulatory system disorders, etc. Although tinnitus affects can range from mild to severe, almost one-quarter of those with tinnitus describe their tinnitus as disabling or nearly disabling/incapacitating, deteriorate the quality of a person's life, and can drastically impact sleep quality. Tinnitus can be particularly debilitating in silent or crowed environments.

Tinnitus has a particularly high prevalence in hearing-impaired persons and electrical stimulation of the inner ear, through for instance cochlear implant, has shown promising results on tinnitus relief and can be considered as a tinnitus management solution. For example, a large number of cochlear implant users experience tinnitus reduction after cochlear implant activation. Although this particular population may not suffer from tinnitus when the cochlear implant is activated/on (e.g., delivering electrical stimulation to evoke hearing percepts), these users can still experience tinnitus when the cochlear implant is switched off and/or idle (e.g., in quiet environments). Most often, this situation occurs at nighttime when the cochlear implant user is attempting to go to sleep, where his/her cochlear implant is deactivated (e.g., switched off and/or in an idle state such that the cochlear implant is generally not delivering signals in a manner to evoke hearing percepts) and the perception of tinnitus sound is highly noticeable. This tinnitus awareness, in turn, causes difficulties in falling asleep.

Conventionally, tinnitus therapies are activated, for example, manually when the user notices the presence of tinnitus and, in general, the particular tinnitus therapy will last for a predetermined of time or until the user deactivates the therapy. Alternatively, conventional tinnitus therapy is activated at certain times of the day (e.g., when the user is attempting to sleep), when the cochlear implant is turned off, etc. These conventional approaches are problematic in that they either require the user to identify the presence of tinnitus and initiate the therapy, or sub-optimally only occur at set times. As such, conventional approaches lack the ability to automatically detect a tinnitus event (e.g., the onset of tinnitus for a user) and dynamically delivery a tinnitus therapy that is optimal for the user for a specific tinnitus event.

As noted above, aspects of the techniques presented herein use machine learning to automatically detect a physiological event, such as a tinnitus event (e.g., perception or presence of tinnitus for a user). Once a physiological event is detected, the techniques presented herein can adjust operation of the cochlear implant, hearing device, or medical device to deliver a treatment/therapy to the user (e.g., deliver a tinnitus therapy), where the attributes of the delivered therapy are selected (adjusted) based on attributes of the detected physiological event, such as severity, timing, physiological data, etc., and the user's determined preferences. Stated differently, the machine learning techniques presented herein allow for the selection of a therapy that is optimized for the specific detected physiological event and for the specific user (e.g., account for the user's therapy preferences).

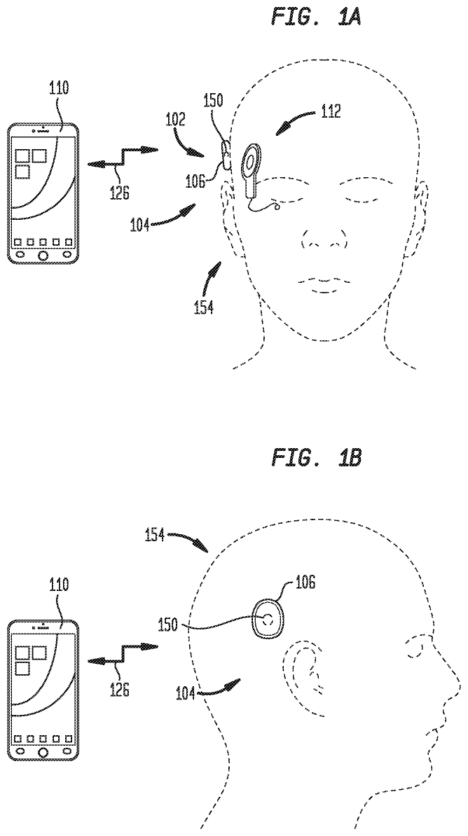

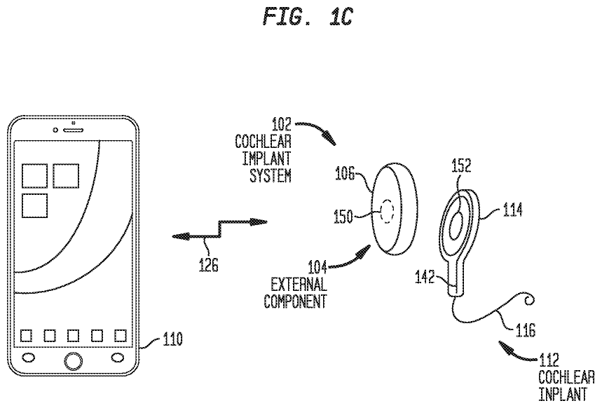

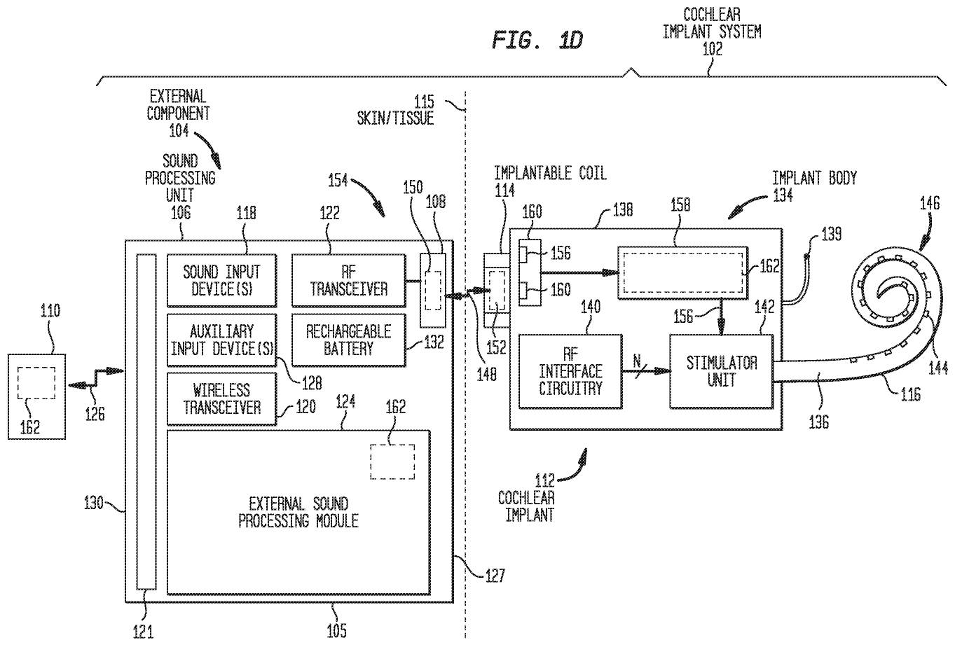

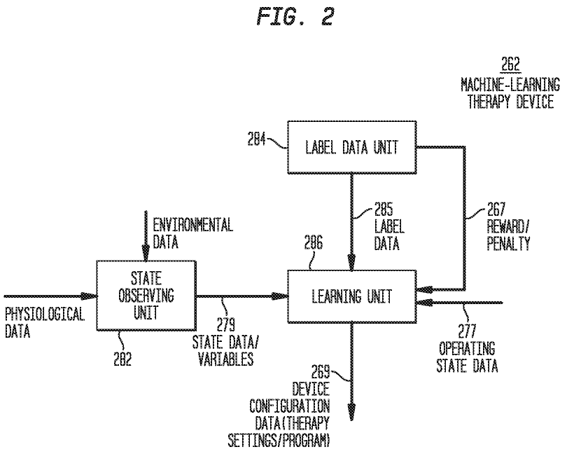

FIGS. 1A-1D illustrates an example cochlear implant system 102 with which aspects of the techniques presented herein can be implemented. The cochlear implant system 102 comprises an external component 104 and an implantable component 112, sometimes referred to as a “cochlear implant.” FIG. 1A illustrates the cochlear implant 112 implanted in the head 154 of a user, while FIG. 1B is a schematic drawing of the external component 104 worn on the head 154 of the user. FIG. 1C is another schematic view of the cochlear implant system 102, while FIG. 1D illustrates further details of the cochlear implant system 102. For ease of description, FIGS. 1A-1D will generally be described together.

Cochlear implant system 102 includes an external component 104 that is configured to be directly or indirectly attached to the body of the user and an implantable component 112 configured to be implanted in the user. In the examples of FIGS. 1A-ID, the external component 104 comprises a sound processing unit 106, while the cochlear implant 112 includes an internal coil 114, an implant body 134, and an elongate stimulating assembly 116 configured to be implanted in the user's cochlea.

In the example of FIGS. 1A-1D, the sound processing unit 106 is an off-the-ear (OTE) sound processing unit, sometimes referred to herein as an OTE component, that is configured to send data and power to the implantable component 112. In general, an OTE sound processing unit is a component having a generally cylindrically shaped housing and which is configured to be magnetically coupled to the user's head (e.g., includes an integrated external magnet 150 configured to be magnetically coupled to an implantable magnet 152 in the implantable component 112). The OTE sound processing unit 106 also includes an integrated external (headpiece) coil 108 that is configured to be inductively coupled to the implantable coil 114.

It is to be appreciated that the OTE sound processing unit 106 is merely illustrative of the external devices that could operate with implantable component 112. For example, in alternative examples, the external component may comprise a behind-the-ear (BTE) sound processing unit or a micro-BTE sound processing unit and a separate external. In general, a BTE sound processing unit comprises a housing that is shaped to be worn on the outer ear of the user and is connected to the separate external coil assembly via a cable, where the external coil assembly is configured to be magnetically and inductively coupled to the implantable coil 114. It is also to be appreciated that alternative external components could be located in the user's ear canal, worn on the body, etc.

As noted above, the cochlear implant system 102 includes the sound processing unit 106 and the cochlear implant 112. However, as described further below, the cochlear implant 112 can operate independently from the sound processing unit 106, for at least a period, to stimulate the user. For example, the cochlear implant 112 can operate in a first general mode, sometimes referred to as an “external hearing mode,” in which the sound processing unit 106 captures sound signals which are then used as the basis for delivering stimulation signals to the user. The cochlear implant 112 can also operate in a second general mode, sometimes referred as an “invisible hearing” mode, in which the sound processing unit 106 is unable to provide sound signals to the cochlear implant 112 (e.g., the sound processing unit 106 is not present, the sound processing unit 106 is powered-off, the sound processing unit 106 is malfunctioning, etc.). As such, in the invisible hearing mode, the cochlear implant 112 captures sound signals itself via implantable sound sensors and then uses those sound signals as the basis for delivering stimulation signals to the user. Further details regarding operation of the cochlear implant 112 in the external hearing mode are provided below, followed by details regarding operation of the cochlear implant 112 in the invisible hearing mode. It is to be appreciated that reference to the external hearing mode and the invisible hearing mode is merely illustrative and that the cochlear implant 112 could also operate in alternative modes.

In FIGS. 1A and 1C, the cochlear implant system 102 is shown with an external device 110. The external device 110 can be a computing device, such as a computer (e.g., laptop, desktop, tablet), a mobile phone, remote control unit, etc. The external device 110 and the cochlear system 102 (e.g., OTE sound processing unit 106 or the cochlear implant 112) wirelessly communicate via a bi-directional communication link 126. The bi-directional communication link 126 may comprise, for example, a short-range communication, such as Bluetooth link, Bluetooth Low Energy (BLE) link, a proprietary link, etc.

Returning to the example of FIGS. 1A-1D, the OTE sound processing unit 106 comprises one or more input devices that are configured to receive input signals (e.g., sound or data signals). The one or more input devices include one or more sound input devices 118 (e.g., one or more external microphones, audio input ports, telecoils, etc.), one or more auxiliary input devices 128 (e.g., audio ports, such as a Direct Audio Input (DAI), data ports, such as a Universal Serial Bus (USB) port, cable port, etc.), and a wireless transmitter/receiver (transceiver) 120 (e.g., for communication with the external device 110). However, it is to be appreciated that one or more input devices may include additional types of input devices and/or less input devices (e.g., the wireless short range radio transceiver 120 and/or one or more auxiliary input devices 128 could be omitted).

The OTE sound processing unit 106 also comprises the external coil 108, a charging coil 130, a closely-coupled transmitter/receiver (RF transceiver) 122, sometimes referred to as or radio-frequency (RF) transceiver 122, at least one rechargeable battery 132, and an external sound processing module 124. The external sound processing module 124 may comprise, for example, one or more processors and a memory device (memory) that includes sound processing logic. The memory device may comprise any one or more of: Non-Volatile Memory (NVM), Ferroelectric Random Access Memory (FRAM), read only memory (ROM), random access memory (RAM), magnetic disk storage media devices, optical storage media devices, flash memory devices, electrical, optical, or other physical/tangible memory storage devices. The one or more processors are, for example, microprocessors or microcontrollers that execute instructions for the sound processing logic stored in memory device.

The implantable component 112 comprises an implant body (main module) 134, a lead region 136, and the intra-cochlear stimulating assembly 116, all configured to be implanted under the skin/tissue (tissue) 115 of the user. The implant body 134 generally comprises a hermetically-sealed housing 138 in which RF interface circuitry 140 and a stimulator unit 142 are disposed. The implant body 134 also includes the internal/implantable coil 114 that is generally external to the housing 138, but which is connected to the transceiver 140 via a hermetic feedthrough (not shown in FIG. 1D).

As noted, stimulating assembly 116 is configured to be at least partially implanted in the user's cochlea. Stimulating assembly 116 includes a plurality of longitudinally spaced intra-cochlear electrical stimulating contacts (electrodes) 144 that collectively form a contact or electrode array 146 for delivery of electrical stimulation (current) to the user's cochlea.

Stimulating assembly 116 extends through an opening in the user's cochlea (e.g., cochleostomy, the round window, etc.) and has a proximal end connected to stimulator unit 142 via lead region 136 and a hermetic feedthrough (not shown in FIG. 1D). Lead region 136 includes a plurality of conductors (wires) that electrically couple the electrodes 144 to the stimulator unit 142. The implantable component 112 also includes an electrode outside of the cochlea, sometimes referred to as the extra-cochlear electrode (ECE) 139.

As noted, the cochlear implant system 102 includes the external coil 108 and the implantable coil 114. The external magnet 152 is fixed relative to the external coil 108 and the implantable magnet 152 is fixed relative to the implantable coil 114. The magnets fixed relative to the external coil 108 and the implantable coil 114 facilitate the operational alignment of the external coil 108 with the implantable coil 114. This operational alignment of the coils enables the external component 104 to transmit data and power to the implantable component 112 via a closely-coupled wireless RF link 148 formed between the external coil 108 with the implantable coil 114. In certain examples, the closely-coupled wireless link 148 is a radio frequency (RF) link. However, various other types of energy transfer, such as infrared (IR), electromagnetic, capacitive and inductive transfer, may be used to transfer the power and/or data from an external component to an implantable component and, as such, FIG. 1D illustrates only one example arrangement.

As noted above, sound processing unit 106 includes the external sound processing module 124. The external sound processing module 124 is configured to convert received input signals (received at one or more of the input devices) into output signals for use in stimulating a first ear of a user (i.e., the external sound processing module 124 is configured to perform sound processing on input signals received at the sound processing unit 106). Stated differently, the one or more processors in the external sound processing module 124 are configured to execute sound processing logic in memory to convert the received input signals into output signals that represent electrical stimulation for delivery to the user.

As noted, FIG. 1D illustrates an embodiment in which the external sound processing module 124 in the sound processing unit 106 generates the output signals. In an alternative embodiment, the sound processing unit 106 can send less processed information (e.g., audio data) to the implantable component 112 and the sound processing operations (e.g., conversion of sounds to output signals) can be performed by a processor within the implantable component 112.

Returning to the specific example of FIG. 1D, the output signals are provided to the RF transceiver 122, which transcutaneously transfers the output signals (e.g., in an encoded manner) to the implantable component 112 via external coil 108 and implantable coil 114. That is, the output signals are received at the RF interface circuitry 140 via implantable coil 114 and provided to the stimulator unit 142. The stimulator unit 142 is configured to utilize the output signals to generate electrical stimulation signals (e.g., current signals) for delivery to the user's cochlea. In this way, cochlear implant system 102 electrically stimulates the user's auditory nerve cells, bypassing absent or defective hair cells that normally transduce acoustic vibrations into neural activity, in a manner that causes the user to perceive one or more components of the received sound signals.

As detailed above, in the external hearing mode the cochlear implant 112 receives processed sound signals from the sound processing unit 106. However, in the invisible hearing mode, the cochlear implant 112 is configured to capture and process sound signals for use in electrically stimulating the user's auditory nerve cells. In particular, as shown in FIG. 1D, the cochlear implant 112 includes a plurality of implantable sound sensors 160 and an implantable sound processing module 158. Similar to the external sound processing module 124, the implantable sound processing module 158 may comprise, for example, one or more processors and a memory device (memory) that includes sound processing logic. The memory device may comprise any one or more of: Non-Volatile Memory (NVM), Ferroelectric Random Access Memory (FRAM), read only memory (ROM), random access memory (RAM), magnetic disk storage media devices, optical storage media devices, flash memory devices, electrical, optical, or other physical/tangible memory storage devices. The one or more processors are, for example, microprocessors or microcontrollers that execute instructions for the sound processing logic stored in memory device.

In the invisible hearing mode, the implantable sound sensors 160 are configured to detect/capture signals (e.g., acoustic sound signals, vibrations, etc.), which are provided to the implantable sound processing module 158. The implantable sound processing module 158 is configured to convert received input signals (received at one or more of the implantable sound sensors 160) into output signals for use in stimulating the first ear of a user (i.e., the processing module 158 is configured to perform sound processing operations). Stated differently, the one or more processors in implantable sound processing module 158 are configured to execute sound processing logic in memory to convert the received input signals into output signals 156 that are provided to the stimulator unit 142. The stimulator unit 142 is configured to utilize the output signals 156 to generate electrical stimulation signals (e.g., current signals) for delivery to the user's cochlea, thereby bypassing the absent or defective hair cells that normally transduce acoustic vibrations into neural activity.

It is to be appreciated that the above description of the so-called external hearing mode and the so-called invisible hearing mode are merely illustrative and that the cochlear implant system 102 could operate differently in different embodiments. For example, in one alternative implementation of the external hearing mode, the cochlear implant 112 could use signals captured by the sound input devices 118 and the implantable sound sensors 160 in generating stimulation signals for delivery to the user.

In the examples of FIGS. 1A-1D, aspects of the techniques presented herein can be performed by one or more components of the cochlear implant system 102, such as the external sound processing module 124, the implantable sound processing module 158, an/or the external device 110, etc. This is generally shown by dashed boxes 162. That is, dashed boxes 162 generally represent potential locations for some or all of the machine-learning therapy device/logic 162 that, when executed, is configured to perform aspects of the techniques presented herein. As noted above, the external sound processing module 124, the implantable sound processing module 158, and/or the external device 111 may comprise, for example, one or more processors and a memory device (memory) that includes all or part of the machine-learning therapy device 162. The memory device may comprise any one or more of: NVM, RAM, FRAM, ROM, magnetic disk storage media devices, optical storage media devices, flash memory devices, electrical, optical, or other physical/tangible memory storage devices. The one or more processors are, for example, microprocessors or microcontrollers that execute instructions for the machine-learning therapy device 162 stored in a memory device.

A machine-learning therapy device presented herein, such as machine-learning therapy device 162, is a functional block (e.g., one or more processors operating based on code, algorithm(s), etc.) that is trained, through a machine-learning process, to detect a physiological event, such as a tinnitus event. A machine-learning therapy device presented herein is further trained, via the same or different machine-learning process, to set/determine a treatment/therapy delivered to the user in response to the detected physiological event, that accounts for the user's preferences and attributes of the physiological event. That is, the techniques presented herein use a machine-learning model to automatically select a user-preferred treatment or therapy to remediate an automatically detected physiological event.

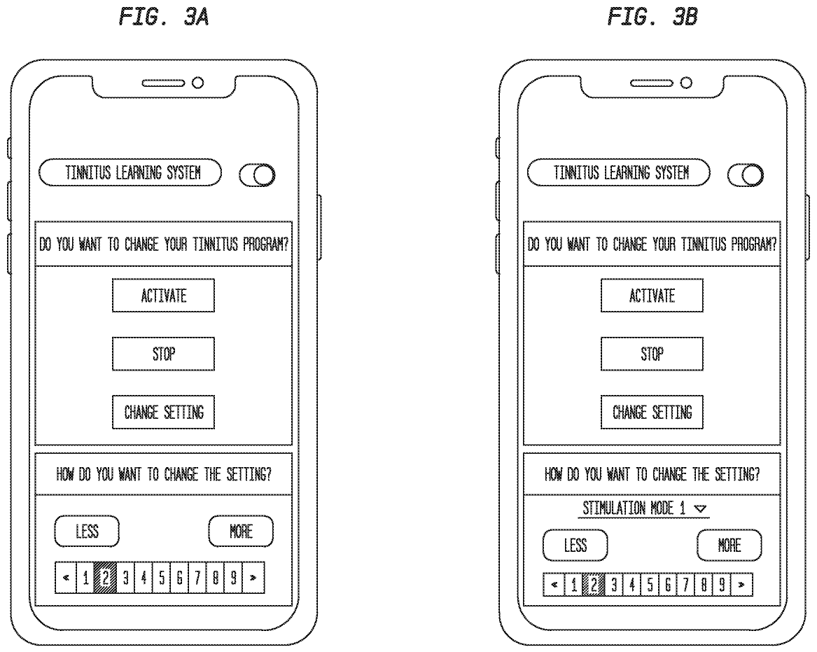

FIG. 2 is a functional block diagram illustrating training and final operation of a machine-learning therapy device 262, in accordance with embodiments presented herein. More specifically, the machine-learning therapy device 262 shown in FIG. 2 includes a state observing unit (state unit) 282, a label data unit 284, and a learning unit 286. As described below, the machine-learning therapy device 262 is configured to generate “device configuration data” 269 (e.g., one or more control outputs) representing at least a selected treatment/therapy for use by the system (implantable medical device) to treat a physiological disorder experienced by the user, where the physiological disorder is manifest as a physiological event. Stated differently, the machine-learning therapy device 262 is configured to determine a preferred therapy for use by the system to treat the user's physiological disorder.

In the example of FIG. 2, the learning unit 286 receives inputs from the state observing unit 282 and the label data unit 284 in order to learn to detect a physiological event, such as a tinnitus event, and to set/determine a therapy delivered to the user in response to the detected physiological event, that accounts for the user's preferences and attributes of the physiological event. In particular, the state observing unit 282 provides state data/variables, represented by arrow 279, to the learning unit 286. The state data 279 includes physiological data, which is data representing the current physiological state of the user. This physiological data can include data representing, for example, heart rate, heart rate variability, skin conductance, neural activity, etc. The physiological data can also include data representing the current stress state of the user. The state data 279 can also include environmental data representing the current ambient environment of the user, such as the current sound environment of the user, current light environment of the user, etc. The learning unit 286 can also receive operating state data 277 representing a current operating state of the system (e.g., tinnitus therapy system/apparatus) and uses the operating state data 277 to set a therapy delivered to the recipient.

In general, the preferred treatment or therapy delivered to a user (e.g., a tinnitus therapy) following detection of a tinnitus event can be subjective for the user and does not follow a linear function corresponding to the state data 279. That is, the device configuration data (selected therapy) 269 cannot be fully predicted based on the state data. Therefore, the label data unit 284 also provides the learning unit 286 with label data, represented by arrow 285, to collect the subjective experience/preferences of the user, which is highly user specific. Stated differently, the label data unit 284 collects subjective user inputs of the user's preferred therapy, which is represented in the label data 285.

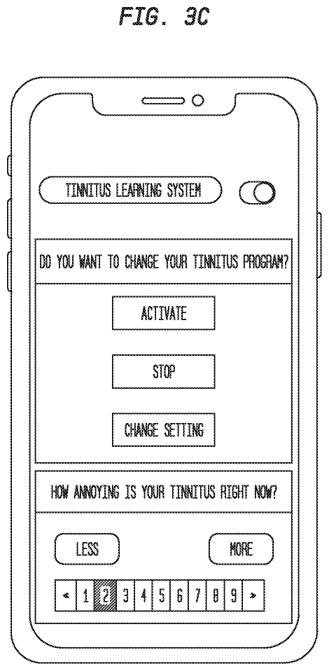

As described further below, the label data 285 can take different forms depending on the stage of the training process. In one example, during the first and second training phases, the user notifies the system when he/she wants to change the therapy setting and will grade his/her subjective need via a user interface (as shown in FIGS. 3A, 3B, and 3C). That is, the label data 285 can represent both a preferred therapy and a subjective ranking/grading of a severity of a physiological event. Through machine-learning techniques, the learning unit 286 correlates the state data 279 and the label data 285, over time, to develop the ability to automatically detect the occurrence of a specific physiological event and to automatically select a preferred therapy for the user, given the specific attributes of the detected physiological event and the user's subjective preferences.

As noted, the label data unit 284 can be a dynamic and progressive unit that collects label data differently depending on the phase of training/use. For instance, in a first example training phase (initial phase), the label data 285 is data collected by the label data unit 284 in real-time. For example, the user is asked/instructed to notify the system when he/she wants to change/optimize the treatment based on his/her subjective input. In other words, the label data 285 can comprise a real-time selection of a preferred therapy in the presence of a physiological event. During this phase, the learning unit 286 is trained to determine which state data 279 represents a specific physiological event and how the user prefers to treat that specific physiological event. Label data 285 collected in real-time is sometimes referred to herein as “real-time event reporting data” as it indicates the real-time subjective feedback of the user in relation to one or more of the onset of a physiological event (e.g., subjective ranking/grading of a severity of a physiological event), a preferred therapy to remediate the physiological event, and/or other real-time information. Label data 285 collected in real-time within a tinnitus therapy system is sometimes referred to as “real-time tinnitus event reporting data.”

In a second example training phase (advanced training phase), the learning unit 286 builds upon the training of the first phase and operates to detect physiological events and selects therapies for treatment of the physiological events over a period of time. However, this is a form semi-supervised learning where the user is asked to confirm or deny the therapy selections made by the system during the time period. More specifically, in the second training phase, the label data 285 collected by the label data unit 284 is retrospective data corresponding to a previous period of time during which the system made selections of preferred therapies and/or therapy changes. For example, the user is asked retrospectively to evaluate therapies automatically selected/adapted by the system during the previous hour, day, etc. In other words, the label data 285 can comprise a retrospective confirmation or evaluation of one or more therapies automatically selected by the system in the presence of a physiological event. Label data 285 collected retrospectively is sometimes referred to herein as “retrospective event reporting data” as it indicates the retrospective subjective preferences of the user in relation to the event detection and/or therapy selections made by the learning unit 286. Label data 285 collected retrospectively within a tinnitus therapy system is sometimes referred to as “retrospective tinnitus event reporting data.”

In a third example training phase (final training phase), the label data 285 collected by the label data unit 284 is data generated automatically based on feedback history (e.g., prior user selections, including real-time event reporting data and retrospective event reporting data) and the user does not necessarily provide any manual inputs. In other words, in this phase, sometimes referred to herein as the automated-operation phase, the label data 285 is generated automatically based on the prior training phases.

Although the user is not asked to give inputs to the system during the automated-operation phase, such inputs can still be provided, as needed, either in real-time or retrospectively. The entry of a user input at this stage results in a change to the feedback/training history (e.g., the feedback history updated if the user notifies the system of a therapy change). In certain examples, the user validation can operate as a reward/penalty input 267 for adaption of the machine-learning process (e.g., adaption of the learning unit 286). Label data 285 generated automatically based on feedback history is sometimes referred to herein as “historical event reporting data” as it is built upon prior real-time and retrospective subjective preferences of the user in relation to the event detection and/or therapy selections. Label data 285 collected retrospectively within a tinnitus therapy system is sometimes referred to as “historical tinnitus event reporting data.”

As described above, the learning unit 286 generates the device configuration data 269 from the state data 279, the label data 285, and, in certain examples, the operating state data 277. Also as noted above, the label data 285 can be progressively changed, over time, so as to decrease in the level of involvement and awareness of the user to the selection of a therapy at a given time. As noted, during the final automated-operation phase, the user does not need to notify the system of a physiological event or to change the selected therapy (e.g., operating state of his/her tinnitus management program) because the system automatically identifies the physiological event and selects the user's preferred therapy for treatment of the physiological event based on the historical training data. It is to be appreciated that the above three training phases are merely illustrative and that the techniques presented herein can use other training phases to train a system to detect a physiological event and selects the user's preferred therapy for treatment of the physiological event.

As noted above, FIGS. 3A, 3B, and 3C are example user interfaces that can be used in the above or other example training phases to provide inputs that result in the generation of label data specifically in relation to tinnitus. More specifically, the interfaces shown in FIGS. 3A and/or 3B could be used during the first training phase to activate, stop, or change a tinnitus therapy. The interface shown in FIG. 3C could be used, for example, to provide an indication of the severity of a tinnitus event. It is to be appreciated that these three interfaces are merely illustrative.

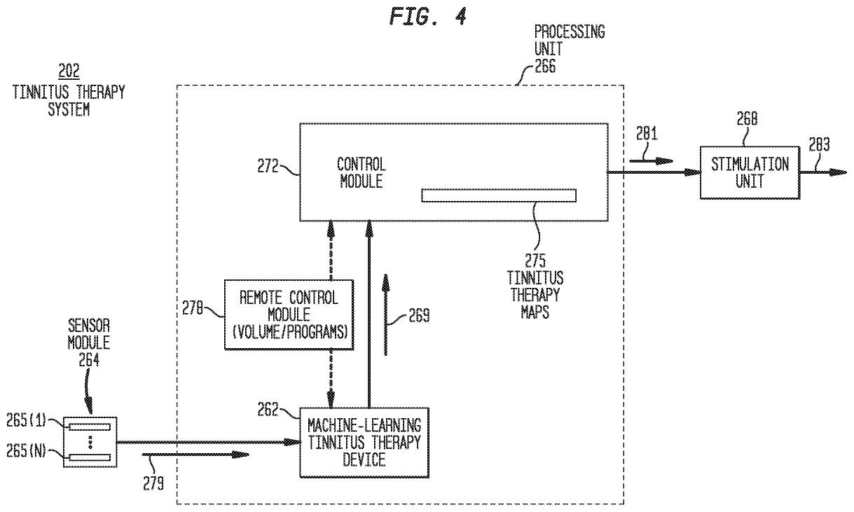

The techniques presented herein can be used to treat a number of different physiological disorders, including inner ear disorders such as vertigo, dizziness, and tinnitus, pain disorders, etc., and the techniques can be implemented by a variety of types of systems. FIG. 4 illustrates a specific use of the techniques presented to select a preferred tinnitus therapy for a user. That is, FIG. 4 is a functional block diagram illustrating an example tinnitus therapy system 202 configured with a machine-learning therapy device, such as machine-learning therapy device 262, for automated selection of tinnitus therapies in response to detected tinnitus events. The tinnitus therapy system 202 could be a stand-alone implantable tinnitus therapy device, incorporated as part of an auditory prosthesis, such as a cochlear implant, bone conduction device, middle ear auditory prosthesis, direct acoustic stimulator, auditory brain stimulator, etc.

It is to be appreciated that the functional blocks illustrated in FIG. 4 can be implemented across one or more different devices or components that can be implanted in, or external to, the body of a user. The tinnitus therapy system 202 can comprise or be a component of, for example, a medical device system (e.g., a cochlear implant system), a computing device, a consumer electronic device, etc. Moreover, as used herein, the term “user” is used to generic refer to any user of a tinnitus therapy system, such as tinnitus therapy system 202, who suffers from tinnitus. The user can also suffer from hearing impairments or physiological disorders other than tinnitus.

As shown, the tinnitus therapy system 202 comprises a sensor unit 264, a processing unit 266, and a stimulation unit 268. Again, the sensor unit 264, the processing unit 266, and the stimulation unit 268 can each be implemented across one or more different devices and, as such, the specific configuration shown in FIG. 4 is merely illustrative.

The sensor module 264 comprises a plurality of sensors 265(1)-265(N) that are each configured to capture signals representing one or more of a current physiological state of a user or an ambient/external sound environment of the user. The signals captured by the sensors 265(1)-265(N) are the “state data” or “state variables” 279 (FIG. 2) and can take a number of different forms and can be captured by a number of different sensors. For example, the sensors 265(1)-265(N) can comprise sound sensors (e.g., microphones capturing sound signals), movement sensors (e.g., accelerometers capturing accelerometer signals), body noise sensors, medical sensors, such as electroencephalogram (EEG) sensors (e.g., one or more external or implantable electrodes and one or more associated recording amplifiers configured to record/measure electrical activity in the user's brain), electromyography (EMG) sensors or other muscle or eye movement detector (e.g., one or more external or implantable electrodes and one or more associated recording amplifiers configured to record/measure muscle response or electrical activity in response to a nerve's stimulation of the muscle), photoplethysmography (PPG) sensor (e.g., sensors configured to optically detect volumetric changes in blood in peripheral circulation), electro-oculogram (EOG) sensors, polysomnographic sensors, Magnetoencephalography (MEG) sensors, heart rate sensors, temperature sensors, skin conductance sensors, Functional Near-Infrared Spectroscopy (fNIRS) sensors, etc. (e.g., recording heart rate, blood pressure, temperature, etc.). It is to be appreciated that this list of sensors is merely illustrative and that other sensors can be used in alternative embodiments.

It is to be appreciated that the state data 279 can also include not only the direct sensor signals, but also processed version of the sensor signals. For example, in certain embodiments, the state data 279 can include sound/environmental classification data generated from captured sound signals. In these embodiments, a sound classification module is configured to evaluate/analyze the sound signals and determine the sound class of the sound signals. That is, the sound classification module is configured to use the received sound signals to “classify” the ambient sound environment and/or the sound signals into one or more sound categories (i.e., determine the input signal type). The sound classes/categories may include, but are not limited to, “Speech,” “Noise,” “Speech+Noise,” “Music,” and “Quiet.” The sound classification module can also estimate the signal-to-noise ratio (SNR) of the sound signals. The sound classification module generates sound classification data that can be part of the state data 279.

In one specific example, the state data 279 represents a combination of skin conductance values, heart rate variability values, and accelerometer signals. In another specific example, the state data 279 represents a combination of skin conductance, photoplethysmography (PPG) sensor signals, such as heart rate variability values and blood volume. In yet another specific example, the state data 279 a combination of neurophysiological measurements, such as EEG signals, MEG signals, and fNIRS signals. It is to be appreciated that these specific combinations of sensor outputs as state data 279 are merely illustrative and that any of a number of different combinations of sensor outputs can be used in alternative embodiments.

In FIG. 4, the state data 279 captured by, or generated from, the sensors 265(1)-265(N) are converted into electrical input signals (if not already in an electrical form), which are represented in FIG. 4 by arrow 279. As shown, the state data 279 (electrical input signals) is provided to the machine-learning therapy device 262. Since, in this example, the machine-learning therapy device 262 is used specifically to treat a user's tinnitus, the machine-learning tinnitus therapy device 262 can be referred to as a “machine-learning tinnitus therapy device (e.g., a machine-learning model configured specifically for treatment of tinnitus).

As shown, the processing unit 266 comprises the machine-learning tinnitus therapy device 262, a control module 272, and a remote control module 278. It is to be appreciated that the functional arrangement shown in FIG. 4 is merely illustrative and does not require or imply any specific structural arrangements. The various functional modules shown in FIG. 4 can be implemented in any combination of hardware, software, firmware, etc., and one or more of the modules could be omitted in different embodiments.

The machine-learning tinnitus therapy device 262 uses the state data 279, the label data 285, and potentially the operating state data 277, to determine whether tinnitus is present and, at least in a final or automated-operation phase to generate device configuration data 269, based on this determination, that is used to generate tinnitus therapy signals 283 for delivery to the user. That is, as noted, the device configuration data 269 represent the user's preferred tinnitus therapy settings/program, as determined through a machine-learning process, such as the one described above with reference to FIG. 2.

The control module 272 is configured to use device configuration data 269 to select, set, determine, or otherwise adjust a tinnitus therapy for the user, as a function of the detected tinnitus e.g., implement the appropriate tinnitus therapy for the user, as determined by the machine-learning tinnitus therapy device 262. Stated differently, the tinnitus therapy that is to be provided to the user is specifically determined and adjusted, in real-time, based on the user's state (e.g., stress, specific needs, etc.) in the presence of tinnitus, potentially at different levels, as determined by the machine-learning tinnitus detection device 262. The tinnitus therapy could also be adapted based on the ambient sound environment.

In accordance with embodiments presented herein, the tinnitus therapy includes the delivery of stimulation signals to the user. These stimulation signals, sometimes referred to herein as “tinnitus therapy signals” or “tinnitus relief signals,” are generated by the stimulation unit 268 and are represented in FIG. 4 by arrow 283. The tinnitus therapy signals can have a number of different forms (e.g., electrical stimulation signals, mechanical stimulation signals, acoustic stimulation signals, visual stimulation signals (e.g., for use in neurofeedback), or combinations thereof) and underlying objectives. For example, in certain embodiments, the tinnitus therapy signals 283 can be masking signals that are configured to mask/cover the user's tinnitus symptoms (e.g., expose the user to sounds/noises at a loud enough volume that it partially or completely covers the sound of their tinnitus). In other embodiments, the tinnitus therapy signals 283 can be distraction signals that are configured to divert the user's attention from the sound of tinnitus. In other embodiments, the tinnitus therapy signals 283 can be habituation signals that are configured to assist the user's brain in reclassifying tinnitus as an unimportant sound that can be consciously ignored. In still other embodiments, the tinnitus therapy signals 283 can be neuromodulation signals that are configured to minimize the neural hyperactivity thought to be the underlying cause of tinnitus. In certain embodiments, the tinnitus therapy signals 283 can be any combination of masking signals, distraction signals, habituation signals, and/or neuromodulation signals.

As noted, in the example of FIG. 4, the tinnitus therapy system 202 includes the simulation unit 268 that is configured to generate the tinnitus therapy signals 283, whether configured for masking, distraction, habituation, and/or neuromodulation purposes. The simulation unit 268 operates based on tinnitus therapy control signals 281 from the control module 272.

The tinnitus therapy control signals 281 can dictate a number of different attributes/parameters for the tinnitus therapy signals 283. For example, the control signals 281 can be such that the tinnitus therapy signals 283 will be pure tone signals, multi tone signals, broadband noise, narrowband noise, low-pass filtered signals, high-pass filtered signals, band-pass filter signals, predetermined recordings, etc. The tinnitus therapy control signals 281 can also set modulations in the tinnitus therapy signals 283, transitions, etc. It is to be appreciated that these specific parameters are merely illustrative, and that the tinnitus therapy signals 283 can have any of a number of different forms.

As described elsewhere herein, the tinnitus therapy signals 283 can be electrical stimulation signals, mechanical stimulation signals, electro-mechanical stimulation signals (e.g., electrical signals and mechanical signals delivered simultaneously or in close temporal proximity to one another), acoustic stimulation signals, electro-acoustic stimulation signals (e.g., electrical signals and acoustic signals delivered simultaneously or in close temporal proximity to one another), etc.

As noted above, the machine-learning tinnitus therapy device 262 is trained to determine the preferred tinnitus therapy. In certain embodiments, the machine-learning tinnitus therapy device 262 can be trained to dynamically adjust a level (amplitude) of the tinnitus therapy signals 283 based on the level of the tinnitus (e.g., from a level of zero to a max level). In other embodiments, the machine-learning tinnitus therapy device 262 can be trained to adjust a frequency or modulation of the tinnitus therapy signals 283. In still further embodiments, the machine-learning tinnitus therapy device 262 can be trained to adjust the type of tinnitus therapy signals 283 (e.g., select one of, or switch between, masking signals, distraction signals, habituation signals, and/or neuromodulation purposes). In the case that the tinnitus therapy signals 283 are electrical stimulation (current) signals, the machine-learning tinnitus therapy device 262 can be trained to adjust one or more of the current level, pulse rate or pulse width of the tinnitus therapy signals 283.

In the specific example of FIG. 4, the control module 272 is configured to store a plurality of different tinnitus therapy maps 275. In general, each of the tinnitus therapy maps 275 is a set/collection of parameters that, when selected, control the generation of the tinnitus therapy signals (e.g., used to generate tinnitus therapy control signals 281). The parameters can control the sound type (e.g., white noise, wave sounds, rain sounds, etc.), fluctuation or modulation rate, amplitude, sound or masker level settings, on/off, pitch settings transition time settings, etc. In operation, different tinnitus therapy maps 275 can be created (e.g., by the software, an audiologist/clinician, through artificial intelligence, etc.) for different situations (i.e., different combinations of body noise classification(s) and environmental classifications). In operation, there will be maps for different therapies, such as specific maps for masking, specific maps for distraction, specific maps for habituation, specific maps for retraining, etc.

In the example of FIG. 4, the machine-learning tinnitus therapy device 262 can be trained to select one of the tinnitus therapy maps 255 for use in generating the tinnitus therapy signals delivered to the user and/or dynamically adjust settings attributes of the tinnitus therapy signals 283. However, it is to be appreciated that the presence of multiple tinnitus maps is merely illustrative and that other embodiments could include one or zero tinnitus maps. For example, the different tinnitus therapy maps 275 could be omitted in alternative embodiments and, instead, the machine-learning tinnitus therapy device 262 is trained to dynamically determine the settings/attributes for tinnitus therapy control signals 281. That is, the specific use of tinnitus therapy maps is merely illustrative and that embodiments presented herein can be implemented without the use of stored tinnitus maps.

In certain examples, selected tinnitus therapy settings can be used to provide tinnitus therapy until the device configuration data 269 from the machine-learning tinnitus therapy device 262 changes in manner that causes the control module 272 to select or adjust the tinnitus therapy. Once the tinnitus therapy adjustment is selected for use, the control module 272 could manage the transition between the settings to avoid unintended issues (e.g., annoyance to the user).

As noted above, the processing unit 266 also comprises a remote control module 278. In certain embodiments, the remote control module 278 can be used to update/adjust, over time, what tinnitus therapy map is selected by the control module 272 based, for example, on user preferences. That is, the remote control module 278 can be used as part of the training process described with reference to FIG. 2 to, for example, receive control data from an external device (e.g., mobile phone) operating with the tinnitus therapy system 202.

As noted above, the tinnitus therapy system 202 is, in certain examples, configured to deliver stimulation signals to the user in order to remediate her tinnitus. In general, the tinnitus therapy can be started when needed and/or ended when not needed anymore. The stimulation signals, referred to herein as tinnitus therapy signals, can be subthreshold signals (e.g., inaudible electrical stimulation signals) or suprathreshold (e.g., audible electrical stimulation signals). As noted, while the tinnitus therapy signals are delivered to the user, one or more attributes/parameters of the tinnitus therapy signals (e.g., amplitude) are dynamically adapted/adjusted based on the control signals 269 from the machine-learning tinnitus detection device 262. In certain embodiments, the tinnitus therapy can be started when needed and/or ended when not needed anymore

In summary, FIG. 4 illustrates an embodiment in which the machine-learning tinnitus detection module 262 is configured to implement an automated learning or adaption process to learn what tinnitus relief settings are optimal for the user (e.g., which signals and parameter settings enable the user to go to sleep the fastest, which signals and parameter settings are preferred by the user, etc.). In certain embodiments, the machine-learned tinnitus detection module 262 is, or includes, a classification function/model configured to generate a classification of whether tinnitus is present or not that is accordingly used to set a therapy. In other embodiments, the machine-learned tinnitus detection module 262 is a regression/continuous function/model and the tinnitus data 271 comprises, for example, a level of the current tinnitus (e.g., a tinnitus level between 0 and 100) and/or other data that is accordingly used to set a therapy. In certain embodiments, the machine-learned tinnitus detection module 262 includes multiple levels that perform classification and regression.

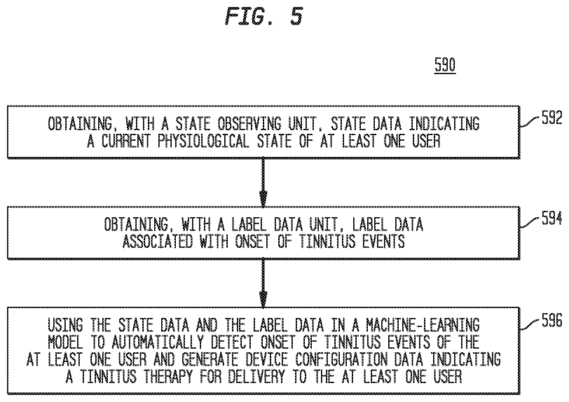

FIG. 5 is a flowchart of an example method 590 for treating tinnitus events using machine learning, in accordance with certain embodiments presented herein. Method 590 begins at 592 where a state observing unit obtains state data indicating a current physiological state of at least one user. At 594, a label data unit obtains label data associated with onset of tinnitus events. At 596, a machine-learning model uses the state data and the label data to automatically detect onset of tinnitus events of the at least one user and generate device configuration data indicates a tinnitus therapy for delivery to the at least one user, wherein the state observing unit, the label data unit, and the machine-learning model comprise one or more of logic hardware and a non-transitory computer readable medium storing computer executable code within a tinnitus therapy system.

As described elsewhere herein, the techniques presented herein can be implemented by a number of different implantable medical device systems to treat a number of different physiological disorders, such as other inner ear disorders (e.g., vertigo, dizziness, etc.), pain disorders, etc. For example, the techniques presented herein can be implemented by auditory prosthesis systems that include one or more other types of auditory prostheses, such as middle ear auditory prostheses, bone conduction devices, direct acoustic stimulators, electro-acoustic prostheses, auditory brain stimulators, combinations or variations thereof, etc. The techniques presented herein may also be implemented by dedicated tinnitus therapy devices and tinnitus therapy device systems. In further embodiments, the presented herein may also be implemented by, or used in conjunction with, vestibular devices (e.g., vestibular implants), visual devices (i.e., bionic eyes), sensors, pacemakers, drug delivery systems, defibrillators, functional electrical stimulation devices, catheters, seizure devices (e.g., devices for monitoring and/or treating epileptic events), sleep apnea devices, electroporation devices, etc. The techniques presented herein may also be partially or fully implemented by consumer devices, such as tablet computers, mobile phones, wearable devices, etc.

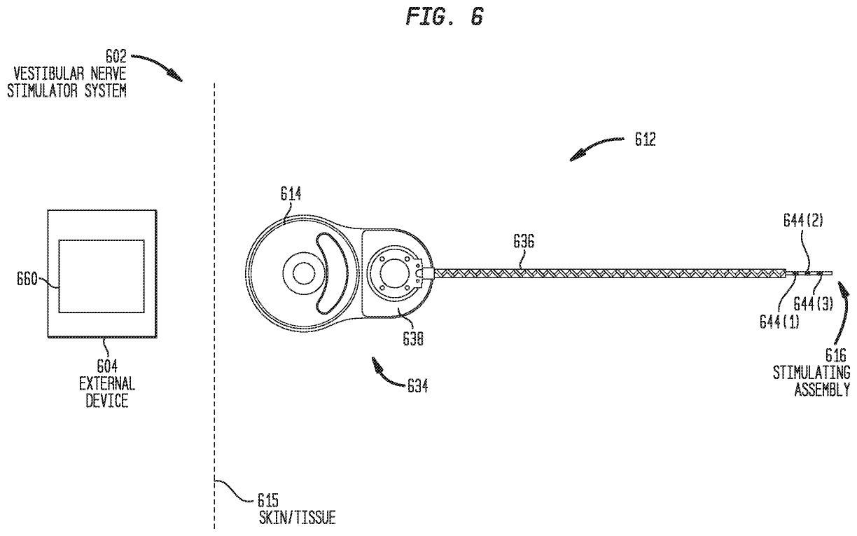

FIG. 6 illustrates an example vestibular stimulator system 602, with which embodiments presented herein can be implemented. As shown, the vestibular stimulator system 602 comprises an implantable component (vestibular stimulator) 612 and an external device/component 604 (e.g., external processing device, battery charger, remote control, etc.). The external device 604 comprises a wireless power transmitter unit 660 that may have an arrangement that is similar to, for example, wireless power transmitter units 360 or 860, described above. As such, the external device 604 is configured to transfer power (and potentially data) to the vestibular stimulator 612,

The vestibular stimulator 612 comprises an implant body (main module) 634, a lead region 636, and a stimulating assembly 616, all configured to be implanted under the skin/tissue (tissue) 615 of the user. The implant body 634 generally comprises a hermetically-sealed housing 638 in which RF interface circuitry, one or more rechargeable batteries, one or more processors, and a stimulator unit are disposed. The implant body 634 also includes an internal/implantable coil 614 that is generally external to the housing 638, but which is connected to the transceiver via a hermetic feedthrough (not shown). In accordance with embodiments presented herein, the external device 604 and/or the implant body 634 could include a machine-learning therapy device, such as machine-learning therapy device 262 described above with reference to FIG. 2.

The stimulating assembly 616 comprises a plurality of electrodes 644 disposed in a carrier member (e.g., a flexible silicone body). In this specific example, the stimulating assembly 616 comprises three (3) stimulation electrodes, referred to as stimulation electrodes 644(1), 644(2), and 644(3). The stimulation electrodes 644(1), 644(2), and 644(3) function as an electrical interface for delivery of electrical stimulation signals to the user's vestibular system.

The stimulating assembly 616 is configured such that a surgeon can implant the stimulating assembly adjacent the user's otolith organs via, for example, the user's oval window. It is to be appreciated that this specific embodiment with three stimulation electrodes is merely illustrative and that the techniques presented herein may be used with stimulating assemblies having different numbers of stimulation electrodes, stimulating assemblies having different lengths, etc.

As should be appreciated, while particular uses of the technology have been illustrated and discussed above, the disclosed technology can be used with a variety of devices in accordance with many examples of the technology. The above discussion is not meant to suggest that the disclosed technology is only suitable for implementation within systems akin to that illustrated in the figures. In general, additional configurations can be used to practice the processes and systems herein and/or some aspects described can be excluded without departing from the processes and systems disclosed herein.

This disclosure described some aspects of the present technology with reference to the accompanying drawings, in which only some of the possible aspects were shown. Other aspects can, however, be embodied in many different forms and should not be construed as limited to the aspects set forth herein. Rather, these aspects were provided so that this disclosure was thorough and complete and fully conveyed the scope of the possible aspects to those skilled in the art.

As should be appreciated, the various aspects (e.g., portions, components, etc.) described with respect to the figures herein are not intended to limit the systems and processes to the particular aspects described. Accordingly, additional configurations can be used to practice the methods and systems herein and/or some aspects described can be excluded without departing from the methods and systems disclosed herein.

Similarly, where steps of a process are disclosed, those steps are described for purposes of illustrating the present methods and systems and are not intended to limit the disclosure to a particular sequence of steps. For example, the steps can be performed in differing order, two or more steps can be performed concurrently, additional steps can be performed, and disclosed steps can be excluded without departing from the present disclosure. Further, the disclosed processes can be repeated.

Although specific aspects were described herein, the scope of the technology is not limited to those specific aspects. One skilled in the art will recognize other aspects or improvements that are within the scope of the present technology. Therefore, the specific structure, acts, or media are disclosed only as illustrative aspects. The scope of the technology is defined by the following claims and any equivalents therein.

It is also to be appreciated that the embodiments presented herein are not mutually exclusive and that the various embodiments may be combined with another in any of a number of different manners.

What is claimed is:

1. A tinnitus therapy apparatus comprising:
a stimulation component configured to deliver stimulation signals to at least one user; and
a machine learning device that detects onset of tinnitus events of the least one user with respect to an external sound environment, wherein the machine learning device includes:
a state observing unit that obtains state data comprising physiological data representing a current physiological state of the at least one user and environmental data,
a label data unit that acquires label data associated with the onset of tinnitus events, and
a learning unit that, by using the state data and the label data, detects the onset of the tinnitus events of the at least one user and generates device configuration data,
wherein the device configuration data indicates a tinnitus therapy for delivery to the at least one user via the stimulation component.

2. The tinnitus therapy apparatus of claim 1, wherein the learning unit receives operating state data representing a current operating state of the tinnitus therapy apparatus, and wherein the learning unit generates the device configuration data further based on the operating state data.

3. The tinnitus therapy apparatus of claim 1, wherein the label data is automatically generated based on historical tinnitus event reporting data representing a history of prior tinnitus events experienced by the at least one user.

4. The tinnitus therapy apparatus of claim 3, wherein the historical tinnitus event reporting data further represents a history of user preferences for treatment of prior tinnitus events experienced by the at least one user.

5. The tinnitus therapy apparatus of claim 3, wherein the historical tinnitus event reporting data is generated based on prior real-time tinnitus event reporting data.

6. The tinnitus therapy apparatus of claim 5, wherein the prior real-time tinnitus event reporting data comprises data representing prior real-time subjective feedback of the at least one user in relation to one or more of the onset of a prior tinnitus event.

7. The tinnitus therapy apparatus of claim 6, wherein the real-time subjective feedback comprises a subjective grading of a severity of at least one prior tinnitus event.

8. The tinnitus therapy apparatus of claim 5, wherein the prior real-time tinnitus event reporting data comprises data representing prior real-time subjective preferences of the at least one user in relation to a preferred therapy to remediate one or more prior tinnitus events.

9. The tinnitus therapy apparatus of claim 5, wherein the historical tinnitus event reporting data is generated based on the prior real-time tinnitus event reporting data and retrospective tinnitus event reporting data.

10. The tinnitus therapy apparatus of claim 9, wherein the retrospective tinnitus event reporting data comprises data representing prior retrospective subjective preferences of the at least one user in relation to a tinnitus therapy selection made in response to a prior tinnitus event.

11. The tinnitus therapy apparatus of claim 1, wherein the label data represents both a preferred tinnitus therapy and a subjective grading of a severity of a tinnitus event experienced by the at least one user.

12. The tinnitus therapy apparatus of claim 1, wherein the physiological data includes data representing at least one of a heart rate or a heart rate variability of the at least one user, a skin conductance of the at least one user, or a neural activity of the at least one user.

13. (canceled)

14. (canceled)

15. The tinnitus therapy apparatus of claim 1, wherein the environmental data includes sound signals captured from an ambient environment of the at least one user or environmental classification data generated from sound signals captured from the ambient environment of the at least one user.

16. (canceled)

17. A method for treating tinnitus events using machine learning, comprising:
obtaining, with a state observing unit, state data indicating a current physiological state of at least one user;
obtaining, with a label data unit, label data associated with onset of tinnitus events; and
using the state data and the label data in a machine-learning model to automatically detect onset of tinnitus events of the at least one user and generate device configuration data indicates a tinnitus therapy for delivery to the at least one user,
wherein the state observing unit, the label data unit, and the machine-learning model comprise one or more of logic hardware and a non-transitory computer readable medium storing computer executable code within a tinnitus therapy system.

18. The method of claim 17, further comprising:
obtaining state data representing an ambient sound environment of the at least one user.

19. The method of claim 18, wherein obtaining state data representing an ambient sound environment of the at least one user comprises:
obtaining sound signals captured from an ambient environment of the at least one user.

20. The method of claim 18, wherein obtaining state data representing an ambient sound environment of the at least one user comprises:
obtaining environmental classification data generated from sound signals captured from an ambient environment of the at least one user.

21. The method of claim 17, further comprising:
receiving operating state data representing a current operating state of the tinnitus therapy system; and
further using the operating state data in the machine-learning model to automatically detect the onset of tinnitus events of the at least one user and generate the device configuration data indicates a tinnitus therapy for delivery to the at least one user.

22. The method of claim 17, wherein obtaining label data associated with the onset of tinnitus events comprises:
automatically generating the label data based on historical tinnitus event reporting data representing a history of prior tinnitus events experienced by the at least one user.

23. The method of claim 22, wherein the historical tinnitus event reporting data further represents a history of user preferences for treatment of prior tinnitus events experienced by the at least one user.

24. The method of claim 22, wherein automatically generating the historical tinnitus event reporting data comprises:
automatically generating the historical tinnitus event reporting data based on prior real-time tinnitus event reporting data.

25. The method of claim 24, wherein the prior real-time tinnitus event reporting data comprises data representing prior real-time subjective feedback of the at least one user in relation to one or more of the onset of a prior tinnitus event.

26. The method of claim 25, wherein the real-time subjective feedback comprises a subjective grading of a severity of at least one prior tinnitus event.

27. The method of claim 24, wherein the prior real-time tinnitus event reporting data comprises data representing prior real-time subjective preferences of the at least one user in relation to a preferred therapy to remediate one or more prior tinnitus events.

28. The method of claim 24, further comprising:
automatically generating the historical tinnitus event reporting data based on the prior real-time tinnitus event reporting data and retrospective tinnitus event reporting data.

29. The method of claim 28, wherein the retrospective tinnitus event reporting data comprises data representing prior retrospective subjective preferences of the at least one user in relation to a tinnitus therapy selection made in response to a prior tinnitus event.

30. The method of claim 17, wherein obtaining label data comprises:
obtaining label data representing both a preferred tinnitus therapy and a subjective grading of a severity of a tinnitus event experienced by the at least one user.

31. The method of claim 17, wherein obtaining label data comprises:
obtaining physiological data representing at least one of a heart rate or a heart rate variability of the at least one user, a skin conductance of the at least one user, or a neural activity of the at least one user.

32-67. (canceled)
